(19) United States
(12) Patent Application Publication
(10) Pub. No.: US 2023/0061702 A1
(43) Pub. Date: Mar. 2, 2023

(54) METHOD FOR RECOVERING PHAS FROM A BIOMASS

(71) Applicant: Meredian, Inc., Bainbridge, GA (US)

(72) Inventors: Carol G. Leggett, Whigham, GA (US); Phillip Van Trump, Decatur, GA (US); Thomas K. Leggett, III, Lexington, KY (US)

(73) Assignee: Meredian, Inc., Bainbridge, GA (US)

(21) Appl. No.: 17/891,435

(22) Filed: Aug. 19, 2022

Related U.S. Application Data
(60) Provisional application No. 63/235,853, filed on Aug. 23, 2021.

(51) Int. Cl. C12P 7/625; C12N 1/20; C12N 1/06; C12N 9/48; C12N 9/22

(52) U.S. Cl. CPC C12P 7/625; C12N 1/20; C12N 1/06; C12N 9/48; C12N 9/22

(57) ABSTRACT

A method for recovering polyhydroxyalkanoates from a biomass is disclosed. According to the method, polynucleotide chains are cleaved by addition of an endonuclease. A lysing agent is used to disrupt cell walls of the microorganism cells and release the intracellular polyhydroxyalkanoates from the cells. Proteins are also degraded by addition of a peptidase. The polyhydroxyalkanoates are then separated from cellular debris of the cells. According to the present disclosure, this method is carried out without the use of organic solvents in the cleaving, lysing, and degrading steps.

### CROSS-REFERENCE TO RELATED APPLICATIONS

This application claims the benefit of the earlier filing date of provisional application 63/235,853, filed Aug. 23, 2021, the disclosure of which is incorporated by reference in its entirety.

### FIELD

This disclosure relates to biodegradable polymeric compositions. More particularly, this disclosure relates to a method for recovering polyhydroxyalkanoates from a biomass of microorganism cells.

### BACKGROUND

Due to environmental concerns associated with conventional petroleum-based polymers, there is a growing interest in alternative polymers which are bio-sourced and/or biodegradable. One class of polymers which are both bio-sourced and biodegradable are polyhydroxyalkanoates. Polyhydroxyalkanoates are typically produced by fermentation of a biomass of bacteria or other microorganisms in a bioreactor. Polyhydroxyalkanoates are synthesized by and accumulate in the cells of the microorganism. To recover the polyhydroxyalkanoates for use, the cells of the microorganisms must be lysed, and the polyhydroxyalkanoates must then be extracted from the lysed cellular material.

Conventionally, organic solvents have been used in this polyhydroxyalkanoate extraction process. While such solvents successfully achieve isolation of the polyhydroxyalkanoates product, the solvents then present additional difficulties. For instance, the organic solvents may themselves raise health, safety, or environmental concerns. Thus, the organic solvents may require special methods of recovery and disposal. They can also result in product contamination.

Consequently, it would be desirable to provide an alternative method to extract polyhydroxyalkanoates from biomass which avoids the use of hazardous organic solvents and is thus more ecologically friendly and requires no special disposal arrangements.

### SUMMARY OF THE INVENTION

In a first aspect, the present disclosure provides a method for recovering polyhydroxyalkanoates from a biomass of microorganism cells containing intracellular polyhydroxyalkanoates. According to one embodiment, the method includes a step of enzymatically treating the microorganism cells of the biomass by: (a) cleaving polynucleotide chains from the microorganism cells by addition of an endonuclease to the biomass; (b) lysing the microorganism cells in the biomass by addition of a lysing agent to the biomass so as to disrupt cell walls of the microorganism cells and release the intracellular polyhydroxyalkanoates from the microorganism cells; and (c) degrading proteins from the microorganism cells by addition of a peptidase, to the biomass. Thereafter, the polyhydroxyalkanoates are separated from cellular debris of the microorganism cells. Importantly, according to the present disclosure, this method is carried out without the use of organic solvents in the cleaving, lysing, and degrading steps.

According to certain embodiments, the endonuclease is preferably a deoxyribonuclease. In certain other embodiments, the endonuclease is preferably a ribonuclease.

In some embodiments, the lysing agent preferably includes an agent selected from the group consisting of lysozyme, bromelain, papain, trypsin, and mixtures thereof. More preferably, the lysing agent comprises lysozyme. The lysing agent is preferably present in an amount from about 0.01 mg/L to about 8 g/L, preferably from about 0.01 mg/L to about 2 g/L, based on the volume of the biomass. Moreover, in certain embodiments, the lysing agent preferably also includes a non-ionic surfactant and a detergent.

In some instances, the lysing step is preferably carried out at a temperature from about 20° C. to about 70° C. for a period of time from about 20 minutes to about 24 hours.

In certain embodiments, the peptidase preferably includes a protease selected from the group consisting of a serine protease, a neutral metalloproteinase, a cysteine protease, and mixtures thereof. Also, according to certain embodiments, the protein degradation step is preferably carried out at a temperature from about 30° C. to about 65° C. for a period of time from about 20 minutes to about 24 hours.

In certain embodiments, the microorganism cells are preferably bacterial cells. More preferably, the microorganism cells comprise at least one bacterial species selected from the group consisting of the genera Ralstonia, Bacillus, Escherchia, Cupriavidus, Alcaligenes, Wausteria, Aeromonas, and Pseudomonas. A particularly preferred species is E. coli.

In some instances, the method also includes an additional step, prior to the enzymatic treatment, of inactivating the microorganism cells in the biomass. This inactivation may be carried out in various ways. For instance, in one embodiment, the cells may be inactivated by exposing the cells to electromagnetic radiation. Alternatively, in another embodiment, the cells may be inactivated by exposing the cells to infrared radiation energy in an amount sufficient to heat the biomass to a temperature of at least 50° C. for a period of time of at least 2 minutes.

In a third embodiment, the cells may be inactivated by injecting steam into the biomass. In still another embodiment, the cells may be inactivated by either: (a) adding one or more acids to the biomass in amount sufficient to establish a biomass pH which is less than about 6.0; or (b) adding one or more bases to the biomass in amount sufficient to establish a biomass pH which is greater than about 8.0; or (c) high pressure (shear) homogenization of the biomass.

In some embodiments, the method also includes an additional step, of bleaching the polyhydroxyalkanoates from the microorganism cells by addition of an oxidizing agent (preferably an oxidizing bleach) to the biomass.

According to certain embodiments, the step of separating the polyhydroxyalkanoates from the cellular debris is preferably carried out by filtration or centrifugation.

In certain embodiments a detergent is also preferably added to the biomass mixture prior to, during, or immediately after the protein degrading step. The detergent may be ionic or nonionic. In some instances, an anionic detergent may be preferred.

In some embodiments, the endonuclease is preferably added to the biomass prior to addition of the lysing agent to the biomass. In other embodiments, the endonuclease and the lysing agent are preferably added to the biomass at the same time.

Also, in certain embodiments, the lysing agent is preferably added to the biomass prior to addition of the peptidase to the biomass. In other embodiments, the lysing agent and the peptidase are preferably added to the biomass at the same time.

### DETAILED DESCRIPTION

In a first aspect, the present disclosure provides a method for recovering polyhydroxyalkanoates from a biomass of microorganism cells containing intracellular polyhydroxyalkanoates.

Polyhydroxyalkanoates are a class of polymers which are both bio-sourced and biodegradable and find increasing commercial interest in a variety of applications. In some instances, polyhydroxyalkanoates recovered according to the present disclosure may be made up of homopolymers, such as polyhydroxybutyrate. In other instances, the polyhydroxyalkanoates be made up of copolymers or even terpolymers formed from differing hydroxyalkanoates monomers.

For instance, in some embodiments, the polyhydroxyalkanoates may be a copolymer made up of from about 75 to about 99.9 mole percent monomer repeat units of 3-hydroxybutyrate and from about 0.1 to about 25 mole percent monomer repeat units of a second hydoxyalkanoate having from 5 to 12 carbon atoms, such as a hydroxyhexanoate hydroxyoctanoate, hydroxydecanoate, hydroxydodecanoate, or hydroxytetradecanoate. More preferably, the second hydoxyalkanoate is 3-hydroxyhexanoate.

In other embodiments, the polyhydroxyalkanoates may be a terpolymer made up of from about 75 to about 99.9 mole percent monomer repeat units of 3-hydroxybutyrate and from about 0.1 to about 25 mole percent monomer repeat units of 3-hydroxyhexanoate, and from about 0.1 to about 25 mole percent monomer repeat units of a third 3-hydoxyalkanoate having from 5 to 12 carbon atoms.

The polyhydroxyalkanoates are typically produced by fermentation of a biomass of microorganisms in a bioreactor. In certain embodiments, the microorganism cells are preferably bacterial cells. More preferably, the microorganism comprises at least one bacterial species selected from the group consisting of the genera Ralstonia, Bacillus, Escherchia, Cupriavidus, Alcaligenes, Wausteria, Aeromonas, and Pseudomonas. A particularly preferred species is E. coli.

In other embodiments, the microorganism comprises at least one species selected from the group consisting of Rhodococcus sp., Bacillus sp., Cupriavidus sp., Aeromonas sp., Pseudomonas sp., Ralstonia sp., Alcaligenes sp., Wausteria sp., Azotobacter sp., Halococcus sp., Halorubrum sp., Halopiger sp., Haloarcula sp., Halomonas sp., Haloferax sp., Halostagnicola sp., Haloterrigena, Halobiforma sp., Halobacterium sp., Natrinema sp., Natronobacterium sp., Natronococcus sp., Halogranum sp., Burkholderia sp., Thiococcus sp., Sinorhizobium sp., Methylobacterium sp., Zobellella sp., Clostridium sp., Salmonella sp., Chloroflexus sp., Shimwellia sp., Klebsiella sp., Azohydromonas sp., Vibrio sp.

The polyhydroxyalkanoates are synthesized by and accumulate in the cells of the microorganism. Thus, to recover the polyhydroxyalkanoates for use, the cells of the microorganisms must be lysed, and the polyhydroxyalkanoates must then be extracted from the lysed cellular material.

In some embodiments of the present disclosure, the method will include an initial step of inactivating the microorganism cells in the biomass prior to any other recovery steps. The inactivation of the biomass cells may be carried out in various ways.

For instance, the biomass cells may be inactivated by exposing the cells to electromagnetic radiation, such as gamma rays, X rays, ultraviolet radiation or infrared radiation for a period of time sufficient to inactivate the cells. Typically, this will range from about 10 seconds to about 30 minutes. In some instances, the biomass cells may be inactivated by exposing the cells to infrared radiation energy in an amount sufficient to heat the biomass to a temperature of at least 50° C. for a period of time of at least 2 minutes.

In an alternative embodiment, the cells may be inactivated by injecting steam into the biomass. In still another embodiment, the cells may be inactivated by either: (a) adding one or more acids to the biomass in amount sufficient to establish a biomass pH which is less than about 6.0; or (b) adding one or more bases to the biomass in amount sufficient to establish a biomass pH which is greater than about 8.0; or (c) high pressure (shear) homogenization of the biomass.

Following any cell inactivation step, the method includes a step of enzymatically treating the microorganism cells of the biomass. This enzymatic treatment includes at least three parts: (a) cleaving polynucleotide chains from the microorganism cells by addition of an endonuclease to the biomass; (b) lysing the microorganism cells in the biomass by addition of a lysing agent to the biomass so as to disrupt cell walls of the microorganism cells and release the intracellular polyhydroxyalkanoates from the microorganism cells; and (c) degrading proteins from the microorganism cells by addition of a peptidase, to the biomass.

In general, it is believed that the three aforementioned parts of the enzymatic treatment (that is, cleaving of polynucleotide chains, lysing of cells, and degrading of proteins) may be carried out in any order in accordance with the method of the present disclosure. In fact, in some instances, two or even all three of these parts of the enzymatic treatment may be carried out at the same time.

For instance, in accordance with certain embodiments of the present disclosure, the endonuclease may be added to the biomass prior to addition of the lysing agent to the biomass. In other embodiments, however, the endonuclease and the lysing agent may be added to the biomass at the same time.

Also, in accordance with certain embodiments of the present disclosure, the lysing agent may be added to the biomass prior to addition of the peptidase to the biomass. In other embodiments, however, the lysing agent and the peptidase may be added to the biomass at the same time.

In certain embodiments of the present disclosure, the endonuclease addition may be carried out first, then the lysing agent addition, and then the peptidase addition.

Again, the enzymatic treatment of the present method includes a step of cleaving polynucleotide chains from the microorganism cells by addition of an endonuclease to the biomass.

The present inventors have found that the release of substantially complete cellular polynucleotide chains from cells in conventional polyhydroxyalkanoates recovery methods leads to a dramatic increase in the viscosity of the biomass mixture. These high viscosities make recovery of the polyhydroxyalkanoates more difficult. However, by adding an endonuclease at this point in the process to cleave the polynucleotide chains into smaller fragments, the inventors have found that a lower viscosity for the biomass mixture may be maintained, thus facilitating recovery of the polyhydroxyalkanoates.

In some instances, this endonuclease may be a deoxyribonuclease, such as endodeoxyribonuclease, exodeoxyribonuclease, or micrococcal nuclease. Alternatively, the endonuclease may be a ribonuclease, such endoribonuclease or exoribonucleases. Moreover, in some embodiments, multiple deoxyribonucleases may be used in the cleaving step, or multiple ribonucleases, or a combination of deoxyribonucleases and ribonucleases.

Typically, the cleaving step is carried out by combining the endonuclease(s) with the biomass of cells in an aqueous mixture, and without any organic solvents. The endonuclease(s) are typically added in an amount from about 0.001 microgram/gram to about 5 milligrams/gram, based on the weight of the biomass being treated.

Water may also be added to the mixture to adjust the total solids content in the mixture. Generally, the total solids content is from about 1 to about 30 weight percent, preferably from about 5 to about 25 weight percent, and more preferably from about 10 to about 15 weight percent.

In general, the biomass and the endonuclease(s) are combining and stirred for a period of time from about 20 minutes to about 24 hours at a temperature from about 30° C. to about 60° C., during which time the endonuclease(s) enzymatically cleave polynucleotide chains from the microorganism cells. Conveniently, this step may be carried out in the same bioreactor in which the biomass is originally fermented, or in another suitable reactor or mixing vessel.

The enzymatic treatment of the present disclosure also includes a lysing step. During this step, a lysing agent is added to the biomass so as to disrupt cell walls of the microorganism cells and release the intracellular polyhydroxyalkanoates from the microorganism cells.

In some embodiments, the lysing agent includes an N-acetyl-muramide glycanhydrolase. Preferably the lysing agent includes a muramidase, such as lysozyme, amidase, glucosaminidase, lytic transglycosylase, and peptidoglycan hydrolase. In other embodiments, the lysing agent may be bromelain, papain, and/or trypsin. More preferably the lysing agent is an agent selected from the group consisting of lysozyme, bromelain, papain, trypsin, and mixtures thereof, in an amount from about 0.01 mg/L to about 8 g/L, based on the volume of the biomass. Even more preferably, the lysing agent is lysozyme. Also, the amount of the lysing agent is more preferably from about 0.01 g/L to about 2 g/L, based on the volume of the biomass

In some instances, the lysing agent may also include a non-ionic surfactant and/or a detergent to increase cell membrane degradation. For instance, the lysing agent may include a non-ionic surfactant such as polymer of polyethylene glycol and/or polypropylene glycol, or polyhydric acids, typically in an amount from about 0.01 weight percent to about 2 weight percent, based on the weight of the biomass. The lysing agent may include a detergent such as a polysorbate (e.g. Tween 20 or Tween 80), Triton, an ethoxylated alcohol (e.g., Tergitol), typically in an amount from about 0.01 weight percent to about 5 weight percent, based on the weight of the biomass.

In general, the lysing is carried out by combining the lysing agent with the biomass of cells in an aqueous mixture, and without any organic solvents. The biomass and the lysing agent are typically combined and stirred for a period of time from about 20 minutes to about 24 hours, preferably from about 2 to 4 hours, at a temperature from about 20° C. to about 70° C. Preferably the temperature is from about 30° C. to about 65° C., and more preferably about 37° C. to about 60° C. During this time, the lysing agent acts to enzymatically disrupt cell walls of the microorganism cells and release the intracellular polyhydroxyalkanoates from the microorganism cells.

As with the cleaving step, this step may be carried out in the same bioreactor in which the biomass is originally fermented, or in another suitable reactor or mixing vessel.

The enzymatic treatment of the present disclosure also includes a protein degrading step. During this step, a peptidase is added to the biomass in order to degrade proteins from the microorganism cells.

Similar to the polynucleotide chains discussed above, the inventors have found that release of large protein structures from the biomass cells also leads to significant increase in the viscosity of the biomass mixture, thereby making recovery of the polyhydroxyalkanoates more difficult. By addition of the peptidase to degrade proteins from the cells into smaller fragments, the inventors have found that a lower viscosity for the biomass mixture may be maintained, thus facilitating recovery of the polyhydroxyalkanoates.

In certain embodiments, the peptidase may include a protease selected from the group consisting of a serine protease, a neutral metalloproteinase, a cysteine protease, and mixtures thereof. A combination of peptidases may also be used. Typically, the protein degrading step is carried out by combining the peptidase(s) with the biomass of cells in an aqueous mixture, and without any organic solvents. The peptidase(s) are typically added in an amount from about 0.01 weight percent to about 5 weight percent, based on the weight of the biomass being treated.

In some instances, a detergent is added to the biomass mixture during, or shortly before or after, the protein degrading step. The detergent may be ionic or nonionic. In some instances, an anionic detergent may be preferred. For example, suitable detergents include may be polyethylene glycol sorbitan monolaurate, polyethylene glycol sorbitan monooleate, polyethylene glycol sorbitan monostearate, polyethylene glycol sorbitan palmitate, secondary ethoxylated alcohols, polyethylene tert-octylphenyl ether, or sodium lauryl sulfate, typically in an amount from about 0.01% to about 10% weight percent, based on the weight of the biomass.

In general, the biomass and the peptidase(s) are combined and stirred for a period of time from about 20 minutes to about 24 hours, preferably from about 2 to about 4 hours, at a temperature from about 30° C. to about 65° C., preferably from about 50° C. to about 60° C., more preferably from about 57° C. to about 58° C., during which time the peptidase(s) enzymatically cleave polypeptide chains from the microorganism cells. As with the previous steps, this step may be carried out in the same bioreactor in which the biomass is originally fermented, or in another suitable reactor or mixing vessel.

After the protein degradation, the polyhydroxyalkanoates are physically separated from cellular debris of the microorganism cells. This separation may be carried out by various separatory techniques. For instance, the separation may be carried out by filtration using a plate and frame filter press, or by tangential flow filtration, or by centrifugation.

Prior to the physical separation, a surfactant—preferably a non-ionic surfactant—may optionally be added to the biomass mixture. The surfactant promotes the formation of micelles which aggregation the cellular debris and facilitate separation of the polyhydroxyalkanoates from the remaining cellular debris.

Optionally, the polyhydroxyalkanoates may be bleached with an oxidizing agent such as sodium hypochlorite, hydrogen peroxide, peracetic acid, or ozone—more preferably with hydrogen peroxide—in order to deodorize and whiten the polyhydroxyalkanoates. Typically, this step is carried out by mixing the oxidizing agent with the biomass after the protein degradation step and before the separation step.

Also, the polyhydroxyalkanoates may optionally be treated in final washing step after the filtration or other physical separation. The washing step may be carried out while the polyhydroxyalkanoates are still in the filter cake on the filter press or in a separate tank after the filtration is completed. The wash mixture may include water alone and/or a mixture of water with aliphatic alcohols such as methanol, ethanol, or isopropanol.

The present disclosure is also further illustrated by the following embodiments:

### Embodiment 1

A method for recovering polyhydroxyalkanoates from a biomass of microorganism cells containing intracellular polyhydroxyalkanoates, the method comprising the steps of:
enzymatically treating the microorganism cells of the biomass by:
(a) cleaving polynucleotide chains from the microorganism cells by addition of an endonuclease to the biomass;
(b) lysing the microorganism cells in the biomass by addition of a lysing agent to the biomass so as to disrupt cell walls of the microorganism cells and release the intracellular polyhydroxyalkanoates from the microorganism cells; and
(c) degrading proteins from the microorganism cells by addition of a peptidase to the biomass; and
separating polyhydroxyalkanoates from cellular debris of the microorganism cells,
wherein the method is carried out without the use of organic solvents in the cleaving, lysing, and degrading steps.

### Embodiment 2

The method of Embodiment 1, wherein the endonuclease comprises a deoxyribonuclease.

### Embodiment 3

The method of Embodiment 1, wherein the endonuclease comprises a ribonuclease.

### Embodiment 4

The method of any of the preceding Embodiments, wherein the lysing agent comprises an agent selected from the group consisting of lysozyme, bromelain, papain, trypsin, and mixtures thereof, in an amount from about 0.01 mg/L to about 8 g/L, based on the volume of the biomass.

### Embodiment 5

The method of Embodiment 4, wherein the lysing agent further comprises a non-ionic surfactant and a detergent.

### Embodiment 6

The method of any of the preceding Embodiments, wherein the lysing step is carried out at a temperature from about 20° C. to about 70° C. for a period of time from about 20 minutes to about 24 hours.

### Embodiment 7

The method of any of the preceding Embodiments, wherein the peptidase comprises a protease selected from the group consisting of a serine protease, a neutral metalloproteinase, a cysteine protease, and mixtures thereof.

### Embodiment 8

The method of any of the preceding Embodiments, wherein the protein degradation step is carried out at a temperature from about 30° C. to about 65° C. for a period of time from about 20 minutes to about 24 hours.

### Embodiment 9

The method of any of the preceding Embodiments, wherein the microorganism cells are bacterial cells.

### Embodiment 10

The method of any of the preceding Embodiments, further comprising, prior to the cleaving step, a step of inactivating the microorganism cells in the biomass by exposing the cells to electromagnetic radiation.

### Embodiment 11

The method of any of Embodiments 1-9, further comprising, prior to the cleaving step, a step of inactivating the microorganism cells in the biomass by exposing the cells to infrared radiation energy in an amount sufficient to heat the biomass to a temperature of at least 50° C. for a period of time of at least 2 minutes.

### Embodiment 12

The method of any of Embodiments 1-9, further comprising, prior to the cleaving step, a step of inactivating the microorganism cells in the biomass by injecting steam into the biomass.

### Embodiment 13

The method of any of Embodiments 1-9, further comprising, prior to the cleaving step, a step of inactivating the microorganism cells in the biomass by either: (a) adding one or more acids to the biomass in amount sufficient to establish a biomass pH which is less than about 6.0; or (b) adding one or more bases to the biomass in amount sufficient to establish a biomass pH which is greater than about 8.0.

### Embodiment 14

The method of any of the preceding Embodiments, further comprising, a step of bleaching the polyhydroxyalkanoates from the microorganism cells by addition of an oxidizing agent to the biomass.

### Embodiment 15

The method of any of the preceding Embodiments, wherein the separating of the polyhydroxyalkanoates from the cellular debris is carried out by filtration or centrifugation.

### Embodiment 16

The method of any of the preceding Embodiments, further comprising, addition of a detergent to the biomass mixture prior to, during, or following the protein degrading step.

### Embodiment 17

The method of any of the preceding Embodiments, wherein the endonuclease is added to the biomass prior to addition of the lysing agent to the biomass.

### Embodiment 18

The method of any of the preceding Embodiments, wherein the endonuclease and the lysing agent are added to the biomass at the same time.

### Embodiment 19

The method of any of the preceding Embodiments, wherein the lysing agent is added to the biomass prior to addition of the peptidase to the biomass.

### Embodiment 20

The method of any of the preceding Embodiments, wherein the lysing agent and the peptidase are added to the biomass at the same time.

### EXAMPLES

The following non-limiting examples illustrate various additional aspects of the invention. Unless otherwise indicated, temperatures are in degrees Celsius and percentages are by weight based on the dry weight of the formulation.

### Example 1

In this example, a biomass made up of an aqueous broth of E. Coli cells from a fermenter was enzymatically treated to extract polyalkanoates from the biomass cells. The total volume of the biomass broth treated was about 100 mL, with a dry solids content of about 15 weight percent.

The extraction process was carried out in a fermenter at an initial temperature of about 37° C. and under mild stirring at about 50 rpm. In a first addition step, the following chemicals were added to the biomass broth:

| Chemical Additive | Amount | |
|---|---|---|
| Polysorbate surfactant (Tmaz 20) | 0.408 | mL |
| Nonionic surfactant (Triton X-100) | 0.81675 | mL |
| Lysozyme lysing agent | 19.60425 | mg |
| Endonuclease (MilliporeSigma | 0.06625 | uL |
| Novagen Benzonase Nuclease) | | |

The additives included two surfactants, an endonuclease (C-Lecta Denarase) to cleave polynucleotide chains, and a lysing agent (lysozyme) to disrupt the cells walls in the biomass. After this chemical addition, the biomass broth was stirred for 60 minutes while being maintained at a temperature of about 37° C. and a pH of about 6.2 to allow the endonuclease and the lysozyme to break down the polynucleotide chains and the cell walls respectively.

Next, the temperature of the biomass mixture was increased to about 60° C. and a second chemical addition was made. In this second addition step, the following chemicals were added to the biomass broth:

| Chemical Additive | Amount |
|---|---|
| Protease (Sebrite BP) | 0.654 mL |
| Sodium Laurel Sulfate Surfactant | 0.0981 mL |
| (Calfoam SLS) | |

After this addition, the biomass broth was stirred for an additional 60 minutes while being maintained at a temperature of about 60° C. to allow the protease to break down proteins from the cells of the biomass.

Next, a third chemical addition was made, in which 3.69 grams of hydrogen peroxide was added to the biomass mixture. After this addition, the biomass broth was stirred for an addition 240 minutes while being maintained at a temperature of about 58° C. to allow the hydrogen peroxide to bleach the mixture, thus improving its color and odor.

Finally, the polyalkanoates were separated from the remainder of the biomass by filtration.

The foregoing description of preferred embodiments for this invention have been presented for purposes of illustration and description. They are not intended to be exhaustive or to limit the invention to the precise form disclosed. Obvious modifications or variations are possible in light of the above teachings. The embodiments are chosen and described in an effort to provide the best illustrations of the principles of the invention and its practical application, and to thereby enable one of ordinary skill in the art to utilize the invention in various embodiments and with various modifications as are suited to the particular use contemplated. All such modifications and variations are within the scope of the invention as determined by the appended claims when interpreted in accordance with the breadth to which they are fairly, legally, and equitably entitled.

What is claimed is:

1. A method for recovering polyhydroxyalkanoates from a biomass of microorganism cells containing intracellular polyhydroxyalkanoates, the method comprising the steps of:
enzymatically treating the microorganism cells of the biomass by:
(a) cleaving polynucleotide chains from the microorganism cells by addition of an endonuclease to the biomass;
(b) lysing the microorganism cells in the biomass by addition of a lysing agent to the biomass so as to disrupt cell walls of the microorganism cells and release the intracellular polyhydroxyalkanoates from the microorganism cells; and
(c) degrading proteins from the microorganism cells by addition of a peptidase to the biomass; and
separating polyhydroxyalkanoates from cellular debris of the microorganism cells,
wherein the method is carried out without the use of organic solvents in the cleaving, lysing, and degrading steps.

2. The method of claim 1, wherein the endonuclease comprises a deoxyribonuclease.

3. The method of claim 1, wherein the endonuclease comprises a ribonuclease.

4. The method of claim 1, wherein the lysing agent comprises an agent selected from the group consisting of lysozyme, bromelain, papain, trypsin, and mixtures thereof, in an amount from about 0.01 mg/L to about 8 g/L, based on the volume of the biomass.

5. The method of claim 4, wherein the lysing agent further comprises a non-ionic surfactant and a detergent.

6. The method of claim 1, wherein the lysing step is carried out at a temperature from about 20° C. to about 70° C. for a period of time from about 20 minutes to about 24 hours.

7. The method of claim 1, wherein the peptidase comprises a protease selected from the group consisting of a serine protease, a neutral metalloproteinase, a cysteine protease, and mixtures thereof.

8. The method of claim 1, wherein the protein degradation step is carried out at a temperature from about 30° C. to about 65° C. for a period of time from about 20 minutes to about 24 hours.

9. The method of claim 1, wherein the microorganism cells are bacterial cells.

10. The method of claim 1, further comprising, prior to the cleaving step, a step of inactivating the microorganism cells in the biomass by exposing the cells to electromagnetic radiation.

11. The method of claim 1, further comprising, prior to the cleaving step, a step of inactivating the microorganism cells in the biomass by exposing the cells to infrared radiation energy in an amount sufficient to heat the biomass to a temperature of at least 50° C. for a period of time of at least 2 minutes.

12. The method of claim 1, further comprising, prior to the cleaving step, a step of inactivating the microorganism cells in the biomass by injecting steam into the biomass.

13. The method of claim 1, further comprising, prior to the cleaving step, a step of inactivating the microorganism cells in the biomass by either: (a) adding one or more acids to the biomass in amount sufficient to establish a biomass pH which is less than about 6.0; or (b) adding one or more bases to the biomass in amount sufficient to establish a biomass pH which is greater than about 8.0.

14. The method of claim 1, further comprising, a step of bleaching the polyhydroxyalkanoates from the microorganism cells by addition of an oxidizing agent to the biomass.

15. The method of claim 1, wherein the separating of the polyhydroxyalkanoates from the cellular debris is carried out by filtration or centrifugation.

16. The method of claim 1, further comprising, addition of a detergent to the biomass mixture prior to, during, or following the protein degrading step.

17. The method of claim 1, wherein the endonuclease is added to the biomass prior to addition of the lysing agent to the biomass.

18. The method of claim 1, wherein the endonuclease and the lysing agent are added to the biomass at the same time.

19. The method of claim 1, wherein the lysing agent is added to the biomass prior to addition of the peptidase to the biomass.

20. The method of claim 1, wherein the lysing agent and the peptidase are added to the biomass at the same time.
